(19) United States
(12) Patent Application Publication
(10) Pub. No.: US 2026/0263337 A1
(43) Pub. Date: Sep. 10, 2026

(54) STREAMLINED SKINCARE SYSTEMS AND METHODS

(71) Applicant: USA Cosmetics, Inc., Frisco, TX (US)

(72) Inventor: Miko Kuo, Frisco, TX (US)

(73) Assignee: USA Cosmetics, Inc., Frisco, TX (US)

(21) Appl. No.: 19/313,661

(22) Filed: Aug. 28, 2025

Related U.S. Application Data
(60) Provisional application No. 63/688,892, filed on Aug. 30, 2024.

(51) Int. Cl. A61K 8/365; A61K 8/55; A61K 8/66; A61K 8/68; A61K 8/99; A61Q 19/10

(52) U.S. Cl. CPC A61K 8/365; A61K 8/553; A61K 8/66; A61K 8/68; A61K 8/99; A61Q 19/10

(57) ABSTRACT

A three-step skincare regimen, compositions, and kits configured to provide the benefits of multi-step routines are disclosed. The regimen includes: (i) a multi-purpose cleanser comprising surfactants and emulsifiers effective to remove both oil-based and water-based impurities in a single step, optionally with mild exfoliants; (ii) a treatment essence combining toner, essence, and serum functions to hydrate and deliver active ingredients, optionally with gentle chemical or enzymatic exfoliation; and (iii) a barrier repair cream containing occlusives, barrier lipids, and humectants to moisturize and support skin barrier restoration, functioning as a moisturizer, facial oil, and overnight mask. Formulations may be tailored for different skin types by varying ingredient classes. Kits include the three compositions and instructions for sequential use to cleanse, treat, and seal in moisture, yielding hydrated, smooth, and radiant skin with fewer steps, lower cost, time, and irritation, and reduced product count compared to conventional extended skincare routines.

### CROSS-REFERENCE TO RELATED APPLICATIONS

This application claims the benefit of U.S. Provisional Application No. 63/688,892, filed Aug. 30, 2024. The contents of that provisional application are incorporated by reference herein in their entirety for all purposes.

### FIELD OF THE DISCLOSURE

The present invention relates generally to skincare regimens and compositions. More particularly, embodiments provide a three-step skincare system-comprising a multi-purpose cleanser, a treatment essence, and a barrier repair cream—that collectively deliver the benefits of traditional multi-step routines in a simplified regimen. The invention further encompasses specific formulations for each of the three products used in the three-step skincare system, methods of use for achieving desirable skin qualities (such as hydrated, “glass skin” appearance), and kits combining the three products for integrated use.

### DESCRIPTION OF THE RELATED ART

Multi-step skincare routines (for example, the 8-10 step regimens popularized in Korean beauty trends) have become renowned for producing smooth, even-toned, dewy skin often referred to as “glass skin.” These extensive routines typically involve sequential application of numerous single-purpose products—such as separate oil and foam cleansers, exfoliators, toners, essences, serums, sheet masks, eye creams, moisturizers, and overnight masks. The underlying philosophy is that layering multiple targeted treatments (e.g., hydrators, mild exfoliants, etc.) yields cumulative benefits for skin clarity and glow.

While effective, ten-step routines have significant drawbacks. They demand substantial time and consistency from users every day. Purchasing and maintaining so many products can be expensive and cumbersome. Moreover, layering numerous formulations increases the risk of irritation or breakouts, especially for sensitive skin, due to potential ingredient conflicts or over-treatment.

Indeed, using too many active products at once can overload the skin's barrier, causing redness or acne flare-ups if not carefully managed. There is a growing recognition that “more” is not always better in skincare, and a trend toward “skinimalism” has emerged. This trend emphasizes using fewer, but multifunctional, products that address core skin needs-cleansing, hydration, barrier support, and protection—in a holistic manner. By focusing on quality over quantity, such minimalist routines aim to reduce unnecessary steps while still achieving healthy, glowing skin.

Travel sets and mini-kits offered by skincare brands have attempted to simplify routines, but conventional multi-product kits merely bundle existing single-purpose products in smaller sizes. For example, a typical travel kit might include a mini cleanser, mini toner, mini serum, and mini cream-providing portability but still requiring 4-5 separate steps with distinct formulations. These kits often do not integrate or combine functions; they simply package the traditional regimen in a travel-friendly way.

Some such sets advertise a complete skincare routine in a pouch and claims to eliminate the need for multiple products for convenience. In reality, the user must still apply each of those multiple items in the correct order, meaning the complexity and layering remain. Thus, the fundamental burden of a lengthy routine is not alleviated by mere co-packaging. There remains a need for a genuinely simplified regimen in which each product is thoughtfully formulated to serve multiple roles, thereby reducing the total number of steps without sacrificing skin benefits.

In view of the above, it would be advantageous to provide a streamlined skincare system that condenses the traditional 8-10 steps into just three synergistic steps. Such a system should deliver comparable improvements in skin hydration, smoothness, and barrier function (the hallmarks of the multi-step approach) while minimizing time, cost, and irritation. Additionally, it should address a variety of skin types-including sensitive and acne-prone skin—by using gentle yet effective ingredients. Embodiments of the present invention fulfill these needs by providing a Three-Step Skincare System that replaces the complex layering regimen with three multifunctional formulations: (1) a gentle multi-purpose cleanser that can double-cleanse and lightly exfoliate, (2) a treatment essence that hydrates like a toner while delivering actives like a serum, and (3) a barrier repair cream that moisturizes and locks in treatment overnight akin to an overnight mask. This integrated approach offers users not only the coveted results of a comprehensive routine—the typical “glass skin” dewy glow achieved through hydration and mild exfoliation—but also goes beyond glass skin, delivering balanced tone, refined texture, and long-lasting hydration, redefined here as “The Barefaced Standard” in certain embodiments, all achieved in just three steps, aligning with the growing preference for simplified “skinimalist” skincare.

### DETAILED DESCRIPTION

The invention will now be described in detail through various embodiments and examples, which illustrate the principles of the three-step system. These descriptions are intended to provide scientific grounding and precise guidance for formulating and using the products in certain embodiments, without limiting the invention's scope. It will be appreciated by those skilled in the art that numerous modifications and substitutions can be made (e.g., swapping specific ingredients within a category, adjusting concentrations, or tailoring to particular skin concerns) without departing from the spirit of the invention. Those of ordinary skill in the art will realize that the following description of the present invention is illustrative only and not in any way limiting. Other embodiments of the invention will readily suggest themselves to such skilled persons upon their having the benefit of this disclosure.

The invention in certain embodiments further encompasses specific formulations for each of the three products used in the three-step skincare system, and methods of use for achieving what is herein referred to as The Barefaced Standard—a skin appearance that goes beyond the traditional “glass skin” ideal. This standard is defined by optimal hydration, balanced barrier function, refined texture, and even tone, such that the skin appears naturally radiant, healthy, and makeup-optional. Unlike single-focus approaches, the disclosed system delivers multi-dimensional improvement in skin quality-addressing hydration, tone, smoothness, firmness, and barrier integrity-through a cleansing step, a treatment step, and a barrier protection step. The invention also encompasses kits combining the three products for integrated use, with variations tailored to different skin types and concerns.

In certain embodiments, the invention provides a three-step skincare regimen and related products that together substitute for a traditional multi-step routine. The system is designed to deliver hydration, exfoliation, targeted treatment, and intensive barrier repair in only three applications, thereby streamlining the user's routine without compromising results. In summary, the key aspects of the invention in certain embodiments include: Step 1 Cleansing Composition; Step 2 Treatment Essence Composition; and Step 3 Barrier Repair Cream Composition.

Each composition is defined by combinations of ingredients that perform multiple skincare functions. For example, the cleanser comprises a blend of mild surfactants, emulsifiers, and optional exfoliants to remove impurities (including makeup and sunscreen) in one step while preserving skin hydration. The treatment essence combines humectants, and may include vitamins and actives to simultaneously tone, hydrate, and treat the skin. The barrier cream includes occlusive agents, emollients, and barrier-restoring lipids to provide lasting moisture, reduce trans-epidermal water loss, and support skin barrier function overnight.

Method of Use (Three-Step Regimen): A method is provided for using the above compositions in a coordinated regimen. The method comprises cleansing the skin with the Step 1 multi-purpose cleanser, then applying the Step 2 treatment essence, and finally applying the Step 3 barrier repair cream, typically in that order. Preferably, this regimen is performed twice daily (morning and evening) with adjustments as needed (e.g., a lighter application of the barrier cream during daytime or use of sunscreen on top in the morning). The method effectively replaces the need for separate makeup removers and exfoliants, toners, serums, face masks, facial oils, and the like, by ensuring each step in the three step sequence delivers a broad spectrum of benefits. Through this method, users can achieve and maintain hydrated, plump, and beyond “glass skin” appearances with significantly fewer products and steps than conventional routines.

Skincare Kit/System: The invention, in certain embodiments, encompasses a skincare kit comprising the three products (cleanser, essence, cream) in a single package, optionally accompanied by instructions for their sequential use as a regimen. The kit may be provided in full-size or travel-size units. Unlike prior art travel sets that are merely assortments of disparate products, this kit is an integrated system wherein each component is formulated to work in concert with the others as a comprehensive but minimal routine. The products can be packaged together in a branded set that emphasizes the regimen's simplicity and efficacy. This encourages user adherence and highlights the streamlining advantage of the system—a key selling point for consumers seeking high-quality results without the burden of lengthy routines.

Scientific and Functional Basis: The specification provides detailed ingredient categories, preferred compositions, and scientific rationale supporting each step's function. For example, the cleanser is pH-balanced and sulfate-free to avoid stripping the barrier, and may include enzymatic or gentle chemical exfoliants to perform daily renewal without separate scrubs. The essence is formulated with high levels of humectants (like glycerin and hyaluronic acid) to boost skin moisture content, plus mild exfoliants (including natural exfoliants) or vitamins for exfoliation and brightening, thereby covering steps that would traditionally be done by separate exfoliators and serums. The barrier repair cream is fortified with known barrier lipids (ceramides, cholesterol, fatty acids) and occlusive agents; this combination both replenishes the skin's natural protective lipids and forms a seal over the skin to prevent overnight moisture loss (trans-epidermal water loss, “TEWL”). The inclusion of such ingredients is grounded in dermatological research showing that ceramides help repair impaired skin barrier function by re-structuring damaged lipid arrangements in the stratum corneum, and that occlusives are highly effective in sealing in hydration and reducing TEWL. Furthermore, the barrier cream's design as an overnight mask substitute is supported by known benefits of sleeping masks: by functioning as both a barrier and a sealant, an overnight application locks in the preceding treatments and moisture, allowing active ingredients to penetrate more effectively without evaporating. This overnight occlusion leads to intense hydration and calming of the skin, aiding the skin's natural nighttime repair processes for maximum results by morning.

Adaptability for Skin Types: Preferred embodiments and formulation variations are described to accommodate sensitive skin, aging skin, acne-prone skin, and oily vs. dry skin without losing core functionality. For example, products are preferably free of common irritants (such as heavy fragrances or alcohol) and non-comedogenic, making them suitable for sensitive or acne-prone users. In one embodiment, the Step 2 essence can be provided in a lightweight water-rich format for oily or acne-prone skin, emphasizing water-based humectants and oil-free soothing agents, whereas a dry-skin variant might be a richer fluid with added emollients. Such variations ensure that the three-step system can be customized to individual needs while still drastically simplifying the routine. By using fewer total products, the system inherently lowers the risk of irritation or ingredient conflicts compared to complex routines, a benefit particularly important for reactive skin. Additionally, using multifunctional products means fewer layering interactions-simplifying the chemistry on the skin-which can help prevent acne breakouts and sensitivity flare-ups that sometimes result from over-layering many products.

In summary, the three-step skincare system of the present invention in certain embodiments provides a comprehensive yet minimalist regimen that achieves the effects of a full multi-step routine (double cleansing, hydration, exfoliation, treatment, barrier protection for luminous healthy skin) with only three hybrid formulations. This innovative approach distinguishes over prior art kits or sets by integrating multiple skincare benefits into each product, rather than merely bundling numerous single-purpose products. The result is a user-friendly regimen that delivers what is defined in certain embodiments as “The Barefaced Standard”—skin that is balanced tone, healthy barrier, refined texture, and sustainable skin health-going beyond the traditional “glass skin” look, without the time investment and complexity of multi-step routines. The invention, in certain embodiments, thus meets the demand of modern skincare consumers for efficacy with efficiency, catering to those who desire the high-performance outcomes of K-beauty-style routines in a fraction of the steps.

For clarity, ingredient categories and technical terms used throughout this application are defined as follows (consistent with usage in cosmetic science):

“Humectants” are ingredients that attract and bind water in the skin's outer layers, thereby increasing hydration. Humectants help replenish moisture and often give the skin a plumper, supple appearance. Examples of humectants include glycerin, propanediol, butylene glycol, sorbitol, urea, hyaluronic acid and its salts (like sodium hyaluronate), betaine, panthenol, and certain alpha hydroxy acids (like lactic acid) which have water-attracting properties. In certain embodiments, humectants are used heavily in the Step 2 essence and also included in the cleanser and cream to ensure each step contributes to skin hydration.

“Occlusives” are moisturizing agents that form a protective, breathable layer on the skin's surface to help reduce transepidermal water loss (TEWL). Unlike humectants, occlusives do not directly add water to the skin but work to seal in existing moisture and the hydration drawn in by humectants. In K-beauty formulations, occlusive ingredients are often chosen for their lightweight yet effective barrier properties, ensuring comfort across different skin types. Commonly used examples include plant-derived oils such as camellia oil, rice bran oil, and jojoba oil; fermented oils for enhanced absorption; shea butter for richer formulations; and silicone-based film formers like dimethicone for a smooth, non-greasy finish. These ingredients are selected to protect the skin's moisture barrier while maintaining the soft, fast-absorbing texture preferred in K-beauty routines. In the Step 3 barrier repair cream, occlusives are critical for forming the “sealant” effect akin to an overnight mask, locking in the actives applied in prior steps and maintaining a hydrated environment on the skin throughout the night.

“Emollients” are ingredients that soften and smooth the skin by filling in tiny gaps between dry or damaged skin cells, improving flexibility and comfort. Many occlusives and plant oils also function as emollients. In K-beauty formulations, commonly used examples include fatty acids (such as stearic and palmitic acid), triglyceride oils (like caprylic/capric triglyceride or rice bran oil), squalane, ceramides, and fatty alcohols. Plant-derived and fermented oils, such as camellia or soybean ferment oil, are also favored for their enhanced absorption and skin-conditioning benefits. In the three-step system according to certain embodiments, emollients are present across all steps but vary in type and concentration.

“Barrier-supporting agents” refers to ingredients known to restore, reinforce, or soothe the skin's protective barrier (the stratum corneum). These include lipids naturally found in the skin barrier (such as ceramides, cholesterol, and long-chain free fatty acids) which can replenish depleted components of the barrier. Ceramides, for instance, are crucial intercellular lipids in the stratum corneum that form lamellar structures and prevent water loss; when barrier function is impaired (as in dryness or eczema), topical ceramides help re-structure the lipid arrangement and repair barrier function. Barrier-supporting agents also encompass anti-inflammatory and soothing ingredients that reduce irritation and allow the barrier to rebuild—e.g., panthenol (pro vitamin B5 which accelerates barrier recovery), niacinamide (vitamin B3, which boosts ceramide synthesis and has anti-redness effects), allantoin, madecassoside (from Centella asiatica), bisabolol (from chamomile), and oat extract. The Step 3 cream in certain embodiments is especially enriched with barrier-supporting agents, though the essence may also include some ingredients (like niacinamide or panthenol) to pre-empt irritation from actives and to support overall skin health.

“Exfoliants” are ingredients that help remove dead cells from the skin's surface, promoting smoother texture and better light reflection (hence more radiant skin). Exfoliants can be physical (mechanical) or chemical. Physical exfoliants include fine scrubbing particles (sugar, salt, synthetic microbeads, jojoba beads, etc.) or tools (brushes, muslin cloths) that manually abrade dead skin. Chemical exfoliants are typically acids or enzymes that loosen the bonds between dead cells or dissolve keratin. Common chemical exfoliants are alpha-hydroxy acids (AHAs, such as glycolic acid, lactic acid), beta-hydroxy acids (BHAs like salicylic acid), and poly-hydroxy acids (PHAs like gluconolactone, which are gentler due to larger molecular size). Mild enzymes (e.g., papain from papaya or bromelain from pineapple) also gently digest dead material. Exfoliation in a multi-step routine is often a separate step (e.g., a scrub or acid toner) done a few times weekly. However, mild daily exfoliation can be built into products; for instance, a cleanser formulated with a mild level of exfoliating agents or natural exfoliants, or an essence containing a low level of exfoliating components, designed to gently refine skin texture while remaining suitable for daily use, or an essence with low-dose lactic acid provides continual renewal without harsh over exfoliation. Research indicates that gentle, consistent exfoliation can help maintain skin glow, keep pores clear, and even improve acne over time-provided it's balanced with hydration and not overdone. According to certain embodiments, exfoliant inclusion is calibrated: the Step 1 cleanser may optionally include a low level of BHA (e.g., 0.5% salicylic) or a gentle enzyme to boost cleansing efficacy, and the Step 2 essence may include an AHA or PHA in the 2-5% range or in low-daily use levels for daily smoothing. These choices ensure the three-step regimen contributes to skin renewal comparable to a dedicated exfoliation step, without requiring an extra product. Stronger exfoliants (high-concentration peels or scrubs) can still be used occasionally as a supplement, but the system's core is sufficient for regular maintenance.

Step 1 cleanser may optionally feature naturally derived ingredients with mild skin-renewing activity—e.g., rice or barley extracts, citrus fruit waters-together with, where suitable for the target skin type, low-level chemical exfoliants (such as AHAs, BHAs, or PHAs) to enhance cleansing efficacy. Step 2 essence may incorporate fermented and/or plant-derived extracts plus cosmetic vitamins-including, for example, a stabilized vitamin C derivative like 3-O-ethyl ascorbic acid—to deliver gentle daily smoothing and brightening benefits.

“Glass skin” in the context of skincare is a descriptive term (originating from Korean beauty discourse) for skin that is exceptionally smooth, even-toned, translucent and dewy-essentially, skin so healthy and hydrated that it has a poreless, reflective quality like glass. Achieving this look typically requires diligent hydration and gentle exfoliation to remove any dull, dead skin and to plump up the skin with moisture. The present system is explicitly designed to yield such outcomes: through intensive hydration (Steps 2 and 3) and controlled exfoliation, along with barrier optimization for skin that is both radiant and resilient. Unlike routines that rely on layering numerous specialized serums for a glass skin effect, this invention, in certain embodiments, provides the necessary hydration and luminosity in a simplified format.

With these definitions in mind, detailed embodiments for each step of the system are described below, although higher or lower amounts may also be used depending on formulation needs.

Step 1: Gentle Multi-Purpose Cleanser (Composition and Function)

| Category | Illustrative % Range |
|---|---|
| Emollients/Oils/Esters | 20-35% |
| Surfactants (mild) | 10-20% |
| Co-surfactants/solubilizers | 2-5% |
| Humectants | 3-10% |
| Botanical Extracts/Soothing Actives | 2-8% |
| Mild Exfoliants (optional) | 0.2-2% |
| pH Adjusters/buffers | 0.1-0.5% |
| Preservative system | 0.8-1.0% |
| Rheology modifiers/emulsifiers | 0.3-2% |
| Fragrance (optional) | 0-0.3% |

In certain embodiments, Step 1 of the regimen is a Multi-Purpose Cleanser that doubles as both a first and second cleanser, meaning it is formulated to effectively remove makeup, sunscreen, excess oil, and debris (like an oil cleanser would) and to dissolve water-based impurities, sweat, and dirt (like a foaming cleanser would). In other words, the Step 1 cleanser allows the user to perform the “double cleansing” often recommended in Korean routines with a single product. This significantly streamlines the cleansing process while ensuring the skin is thoroughly clean and prepared for subsequent treatment. Key characteristics and components of the Step 1 cleanser include:

Mild Surfactant Base: The cleanser utilizes sulfate-free, gentle surfactants to avoid harsh stripping of the skin's natural oils. Preferred surfactants include amphoteric and non-ionic types such as cocamidopropyl betaine, decyl glucoside, sodium cocoyl glutamate, or disodium cocoamphodiacetate, often combined with low levels of mild anionic surfactants like sodium lauroyl sarcosinate to enhance cleansing while maintaining gentleness. These provide effective removal of impurities with a mild lather, sufficient to lift debris without compromising the skin barrier. The formulation targets a skin-friendly pH (around 5 to 6) to help maintain the acid mantle, as low-pH cleansers have been shown to better preserve barrier integrity and support the skin microbiome compared to high-pH soaps, which can cause dryness.

Emulsifiers for Makeup Removal: To enable effective removal of oil-based impurities (sebum, long-wear makeup, waterproof sunscreen), the cleanser optionally contains emulsifying agents and/or a small oil phase. For example, it can include polysorbate 20 or PEG-7 glyceryl cocoate, which help solubilize makeup and oils on the skin so they rinse away easily with water. In certain embodiments, the cleanser includes gentle surfactants (such as sodium cocoyl isethionate and cocamidopropyl betaine) combined with a small oil phase (e.g., sunflower seed oil and avocado oil). In some embodiments, the cleanser may be a hybrid oil-to-foam formulation: initially applied as a gel or oil consistency that breaks down makeup, and upon adding water, it emulsifies into a milky or foaming solution that carries away both oil-soluble and water-soluble debris. This mimics the traditional two-step cleansing (oil cleanser followed by foaming cleanser) in one product—a common approach in modern K-beauty to deliver a thorough cleanse without requiring two separate products. In further embodiments, the cleanser may take a variety of initial formats before transforming upon addition of water, including but not limited to balm-to-foam, gel-to-foam, oil-to-foam, cream-to-foam, powder-to-foam, milk-to-foam, or cleansing water-to-foam systems. Each such variant provides a hybrid one-step cleansing action that consolidates the functions of both an oil cleanser and a foaming cleanser.

Hydrating and Soothing Additives: Unlike many cleansers that leave the skin feeling tight, in certain embodiments the cleanser is enriched with humectants (e.g., glycerin, butylene glycol) and soothing agents (such as panthenol and Centella asiatica extract) so that it cleanses without over-drying. Glycerin in the cleanser can deposit onto the skin, helping it stay hydrated even after rinsing. These additions help ensure that the cleanser supports the skin's barrier-aligning with the overall regimen's goal of barrier care at every step.

Optional Mild Exfoliation: In certain embodiments, the cleanser provides a gentle exfoliating action to further streamline the routine. For example, natural extracts commonly used in Korean skincare—such as rice extract, barley extract, and lime fruit water—may offer mild exfoliating and skin-brightening properties by promoting enzymatic or low-level alpha-hydroxy-like effects. These botanical ingredients help slough away dead surface cells without irritation. By integrating such naturally derived, non-acid exfoliants in Step 1, the formula helps maintain smooth skin texture and improves absorption of the Step 2 essence—without requiring a separate scrub step. This aligns with Korean skincare philosophy and dermatological guidance that supports gentle, regular exfoliation over abrasive or infrequent scrubbing to refine texture, unclog pores, and enhance glow.

Non-Comedogenic, Rinse-Clean Feel: The cleanser preferably is formulated to rinse off completely without any pore-clogging residue. Ingredients are selected with acne-prone skin in mind (e.g., avoiding high concentrations of heavy oils or occlusives in the cleanser that might deposit). The product is tested to be non-comedogenic, meaning it should not contribute to blackheads or pimples. Additionally, any emulsifiers or oils used are balanced so that the skin feels clean and fresh after use, not greasy. For instance, if a cleansing oil component is included, it is combined with enough solubilizer that upon rinsing, no oily film remains (a hallmark of a good emulsifying cleanser).

In use, about a dime-sized amount of this cleanser is applied to damp skin and massaged in circular motions. Thanks to the inclusion of emulsifiers and mild scrub action, the user can (if wearing makeup or sunscreen) apply it first to dry face to dissolve makeup, then add a bit of water to lather. The cleanser produces a light foam, cleans thoroughly, and rinses off easily. Skin is left clean but soft, without the “stripped” feeling. Importantly, this single step suffices in place of what conventionally might be two cleansing steps (oil cleanse+foam cleanse) and possibly an extra exfoliation step. The skin is now a clean “blank canvas” prepared to receive the treatment essence.

### Step 2: Treatment Essence (Multifunctional Toner/Serum Hybrid)

Step 2 of the system is a treatment essence, a multi-benefit fluid that combines the roles of a traditional toner, essence, and serum into one product. In classic routines, after cleansing one would apply a toner (to rebalance pH and lightly hydrate), then perhaps an essence (for additional hydration and nutrients), then one or more serums targeting specific concerns (like brightening, anti-aging, or pore refining). Here, the single Step 2 formulation provides hydration, prepares the skin, and delivers active ingredients for treatment, effectively collapsing several layers into one application. The product is called an “essence” to signify its lightweight, fast-absorbing nature similar to Korean essences, but it is enriched with enough actives to also serve as a serum-step. Key aspects include:

Hydration and Skin Prep: The essence is formulated with a high concentration of humectants to saturate the stratum corneum with water. Ingredients like hyaluronic acid (including multiple molecular weights for layered hydration), glycerin, and butylene glycol attract moisture to the skin, immediately plumping and softening it. This hydrated state not only improves the skin's appearance (dewy and smooth) but also makes the skin more permeable to beneficial actives (moist skin is more receptive than dry skin). In this way, the Step 2 essence plays the “toner” role of priming the skin and the “sheet mask” role of deeply hydrating (without the need for an actual mask sheet). Some embodiments might even instruct the user to apply multiple thin layers of the essence (a technique sometimes called the “7-skin method” in K-beauty, where a hydrating toner is layered repeatedly) for extra hydration instead of using a separate sheet mask. Because the product is lightweight, layering is possible if the user desires more hydration, but even one layer provides significant moisture.

Active Treatment Ingredients: The essence includes active skincare agents that address common skin goals—such as brightening dull skin, reducing appearance of pores, calming redness, and providing anti-aging benefits. These actives are chosen to be generally compatible (to avoid requiring separate specialized serums) and suitable for daily use. Some exemplary actives, without limitation, are:

Niacinamide (Vitamin B3)—included at around 2-5%, although higher or lower amounts may also be used depending on formulation needs. Niacinamide is a versatile active known to improve uneven skin tone, reduce pigmentation, refine pore size, and strengthen the skin barrier by increasing ceramide synthesis. It's also anti-inflammatory, which helps with acne and sensitive skin. Because niacinamide is gentle and pairs well with many other ingredients, it's an excellent all-in-one active for a minimalist regimen.

Vitamin C or Derivatives-included for brightening and antioxidant support. A stabilized form like sodium ascorbyl phosphate or ascorbyl glucoside can be used at ~5%, to promote radiance and stimulate collagen without the instability of pure ascorbic acid. This helps give skin a glow and fades faint dark spots over time, substituting for a separate brightening serum.

Mild Exfoliant (AHA/PHA)—As an option, Step 2 may include a gentle concentration of an alpha-hydroxy acid (AHA) such as lactic acid or mandelic acid (around 2-5%) or a polyhydroxy acid (PHA) such as gluconolactone (3-10%), to promote smoothness and radiance. Naturally derived brightening exfoliants, such as Oryza sativa (rice) extract, are also commonly incorporated for an even gentler, enzyme-based resurfacing. These ingredients work to gradually loosen and remove dead skin cells, enhancing skin texture and glow over time without the harshness of traditional exfoliation.

At the low pH of the essence (~pH 5), a modest AHA can function effectively yet gently, eliminating the need for a separate exfoliating toner. Because over-exfoliation can irritate, the formulation balances acids with humectants and soothing agents (and users with very sensitive skin might choose an acid-free version of the essence).

Anti-inflammatory Botanicals: To ensure even active ingredients do not cause irritation, the essence preferably contains calming botanical extracts. Examples include Centella asiatica extract (rich in madecassoside, known to soothe and aid healing), green tea extract (antioxidant and anti-redness), licorice root extract (brightening and anti-irritant), or chamomile (bisabolol). These help modulate any potential irritation from exfoliants or actives, making the product suitable for sensitive skin usage.

Barrier & Brightening Support: In some embodiments, the essence also contains barrier-supportive and brightening actives such as niacinamide or panthenol, which help improve skin tone, reinforce the skin barrier, and maintain hydration. These additions allow the essence to double as a hydration and radiance treatment, reducing the need for multiple separate serums in a routine.

Peptides or Collagen Boosters: In some embodiments, small peptides (like palmitoyl tripeptide-5 or acetyl hexapeptide-8) are included to provide anti-aging benefits (supporting collagen, reducing fine lines). Dr. Engelman, for instance, recommends peptides in overnight treatments for their collagen support; similarly, inclusion of a peptide in this essence can proactively firm the skin over long-term use. Such additions allow the product to take the place of an anti-aging serum in the routine.

Lightweight, Layerable Format: The texture of the essence is ideally a thin gel or viscous liquid that can quickly absorb. It should not be as thick or occlusive as a cream, since it goes under the barrier cream. Formulators might use a gelling agent like Ammonium Acryloyldimethyltaurate/VP Copolymer or Sodium Acryloyldimethyl Taurate Copolymer-which give a lightweight, elegant, slightly viscous texture. These may also provide slight viscosity (so it is not as runny as pure water), making it easier to pat onto the skin. For oily skin versions, the essence can be oil-free, relying purely on water and glycols for slip (such as butylene glycol or methylpropanediol), and lightweight humectants to provide hydration and a smooth application feel. For dry skin versions, a small percentage of a light oil (like squalane or Caprylic/Capric Triglyceride, which are lightweight, non-greasy emollients from coconut oil and glycerin) or a phospholipid liposome could be included to help deliver actives and add a hint of emollience without heaviness. In all cases, the essence is formulated to be non-sticky and fast-absorbing, leaving skin feeling refreshed and bouncy—a sign that it has imparted water and beneficial ingredients without clogging.

Substitution for Multiple Steps: The treatment essence is explicitly designed to replace at least three separate conventional products: (1) a toner, (2) an essence or “softener,” and (3) a treatment serum. It can even stand in for a mild exfoliating product and, partly, for a hydrating sheet mask. In certain embodiments, it may also include cream essence, moisturizer, toner, gel, lotion, or skin booster. This consolidation provides both hydration and concentrated actives in one step, thereby avoiding the layering of multiple serums (which can be confusing or irritating). The formula is balanced such that despite having many functions, it remains cosmetically elegant. Furthermore, by eliminating extraneous fillers (since one product carries the weight of many), it becomes possible to invest in higher-quality ingredients within that single bottle-aligning with the skinimalism philosophy of “fewer but better” products. The user benefits from simplicity: after cleansing, they need only reach for this one treatment essence to address a spectrum of needs, then move to moisturizing.

### Example Formulation-Step 2 Treatment Essence

An example composition for the essence could be as follows, although higher or lower amounts may also be used depending on formulation needs:

| Category | Illustrative % Range |
|---|---|
| Vehicle/solvent | 55-75% |
| Humectants | 8-20% |
| Botanical/ferment extracts | 3-10% |
| Vitamin & peptide actives | 2-5% |
| Light emollients/occlusives | 0-3% |
| Texture/rheology modifiers | 0.1-0.5% |
| Preservative system | 0.5-1.0% |
| pH adjusters/buffers | 0.1-0.3% |
| Optional fragrance/EO | 0-0.2% |

After cleansing, a user would dispense, for example, 3-4 drops (or 1-2 pumps) of this essence into their palms and press it into the face and neck. It spreads easily, absorbing within moments, and leaves a slight dewy finish without any residue. Immediately, the skin feels hydrated (thanks to glycerin, butylene glycol, HA) and soothed (due to Centella and allantoin). The niacinamide and other actives begin to work on brightness and texture with daily use. There is no need for a separate toner—this essence balances and hydrates in one step. The skin is now optimally conditioned to receive the final Step 3, and in fact, the moistness from the essence aids the spreadability of the subsequent cream.

Notably, at this stage in a conventional routine, one might apply multiple toners and serums or even a sheet mask, taking extra 10-20 minutes. In contrast, with a three-step system according to certain embodiments, after the essence, one can proceed directly to the last step, saving time while still ensuring the skin has been amply nourished and treated. The streamlining benefit is clear: the essence delivers concentrated treatment without requiring layering of numerous products, aligning with a minimalist approach that still fulfills the skin's needs.

### Step 3: Barrier Repair Cream (Moisturizer, Occlusive Mask, and Facial Oils in One)

Step 3 is the Barrier Repair Cream, which serves as the final seal and restorative treatment in the regimen. This product is designed to function as a rich moisturizer and an occlusive overnight mask, consolidating what, in traditional routines, might be a moisturizer, a cream, a sleeping pack, and possibly an eye cream and/or a facial oil. The barrier repair cream is applied as the last step to lock in all the hydration and actives applied earlier, and to fortify the skin's natural barrier for sustained moisture and protection.

Key features of the Step 3 barrier repair cream include:

Occlusive Barrier Formation: A primary role of this cream is to form an effective barrier on the skin surface to prevent moisture loss over time (particularly overnight). To this end, the cream contains a blend of occlusive agents such as petrolatum, dimethicone, and natural waxes/butters. Petrolatum, in particular, is highly preferred in some embodiments at about 5-15% of the formula, given that it is recognized as one of the most effective occlusives, able to reduce transepidermal water loss (TEWL) by up to ~99% when used in pure form, although higher or lower amounts may also be used depending on formulation needs. Even at lower concentrations in a cream, petrolatum dramatically decreases water evaporation and helps maintain skin hydration. For users who prefer non-petroleum alternatives, plant-derived occlusives like shea butter, cocoa butter, or lanolin can be employed, as well as high molecular weight silicones (dimethicone at 2-4% can create a breathable barrier). By reducing TEWL, the cream ensures that the moisture provided by the Step 2 essence and additional humectants in the cream remains in the stratum corneum through the night. This mimics the effect of popular “slugging” techniques (where petrolatum is applied as a seal) but in a more elegant, all-in-one product form.

Overnight occlusion has known benefits: It not only deeply hydrates but also enhances active ingredient penetration and effectiveness, and it protects the skin from external irritants or dirt while one sleeps. Users can effectively get the benefit of an overnight sleeping mask by using this barrier cream as the last step.

Barrier Lipid Restoration: Beyond just sealing in moisture, the barrier repair cream actively restores the skin's barrier by supplying skin-identical lipids. Preferably, the cream contains a ceramide complex (for example, a mix of Ceramide NP, Ceramide AP, Ceramide EOP, etc.) in combination with cholesterol and free fatty acids (such as stearic acid, linoleic acid, or palmitic acid). Research supports that topical application of these lipids, in ratios similar to those in the natural skin barrier, can significantly improve barrier function and repair damage. Ceramides specifically help refill the “mortar” between skin cells, reinforcing the lamellar structures that keep the skin impermeable to water loss and irritants. In formulations, ceramides are often used at around 0.5-2% (due to potency and cost), cholesterol similarly around 0.5-3%, and fatty acids around 1-5%, although higher or lower amounts may also be used for all of these, depending on formulation needs. By including these, the cream not only prevents water from escaping but also contributes to the rebuilding of a healthy barrier over time (thus addressing root causes of dryness or sensitivity). This is a distinguishing factor from basic moisturizers-many moisturizers focus on immediate hydration, but a “barrier repair” cream per this invention, in certain embodiments, actively improves the skin's resilience long term through lipid supplementation. The presence of niacinamide in the cream (as an optional component around 2-4%) can further boost the skin's own production of ceramides and barrier lipids, making the combination synergistic.

Intensive Moisture and Emollience: The cream provides rich moisturization through a combination of emollients and humectants. Emollient oils such as squalane, camellia seed oil, rice bran oil, sunflower seed oil, jojoba oil, soybean oil, or phytosqualane can be included to soften the skin and fill in any microscopic cracks in the dry skin surface, immediately improving texture. These oils also help dissolve and deliver oil-soluble actives in the formula (like certain vitamins or botanical extracts). Humectants like glycerin, butylene glycol, or betaine are present in certain embodiments to bind water within the skin-often, the best moisturizers use both humectants and occlusives, as humectants draw in water and occlusives keep it there. For example, glycerin at 3-5% and sodium PCA at 1% can significantly boost the water content of the stratum corneum. Urea is another powerful humectant (and keratolytic in higher amounts) that can be used around 2-5% to hydrate and even gently exfoliate flaky skin.

Overnight Repair Actives: Since this cream will often be used at night as the final step (though it can also serve as a day cream for dry skin, with a lighter layer or under sunscreen), it is an ideal vehicle for overnight actives. During sleep, skin goes into repair mode, and studies show that cell regeneration increases at night. The cream can leverage this by including ingredients like:

Panthenol (Pro-Vitamin B5)—a classic skin-repair ingredient that promotes barrier recovery and has anti-inflammatory effects; it soothes and supports skin regeneration overnight.

Peptides—similar to those in the essence or others like Matrixyl (palmitoyl pentapeptide-4) to encourage collagen renewal and skin firmness during the night.

Antioxidants—such as Vitamin E (tocopherol or tocopheryl acetate) or Coenzyme Q10, to protect the skin from oxidative stress and aid repair from any daytime UV damage. These work well in the lipid-rich environment of a cream.

Botanical oils/extracts that aid barrier and have anti-inflammatory benefits, e.g., sunflower seed oil (rich in linoleic acid and beneficial for maintaining barrier integrity), help ensure that, while the cream is occluding and preventing water loss, it is simultaneously delivering compounds that help heal micro-damage and reduce any irritation from the day's environmental exposure or from active treatments used. Ferment lysates such as Bifida, Saccharomyces, and Lactobacillus, which are known to strengthen the skin's microbiome and calm irritation, may also be used in certain embodiments.

Non-comedogenic and Adjustable Texture: Although this cream is rich by design, it is formulated to be non-comedogenic-using oils and butters that have low comedogenic ratings and formulating with the minimum occlusive load required for efficacy. For example, while coconut oil (highly comedogenic) is avoided, ingredients like shea butter (moderate) might be used in moderation, and non-comedogenic synthetic occlusives like dimethicone are utilized. The product's texture is preferably a cream that absorbs well without a greasy after feel, achieved by using emulsifiers that create a stable emulsion with small oil droplet size. Emulsifiers like glyceryl stearate, cetearyl alcohol (which also serves as a fatty emollient), and Polyglyceryl-3 Methylglucose Distearate (a plant-derived, PEG-free emulsifier and a favorite in clean beauty and EWG-compliant formulations) can create smooth, gentle emulsions ideal for sensitive skin and can be used to blend the oil and water phases. The viscosity can be set to a thick cream or a lighter gel cream depending on target skin type:

Variants for individuals with oily/acne-prone skin still provide barrier protection but feel lighter. They may incorporate mattifying agents (like a bit of silica or porous silica derivatives or lightweight polymer powders such as polymethyl silsesquioxane), which may be included for an elegant, non-chalky finish, so that even those with oily skin can use it overnight without feeling too slick. In fact, even oily/acne-prone individuals benefit from barrier repair at night, since many acne treatments (like betaine salicylate or low-pH salicylic acid (BHA)) compromise the barrier; a non-comedogenic barrier cream can reduce irritation and improve tolerance of such treatments.

Eye Area Use: Because the routine does not include a separate eye cream, the barrier repair cream is formulated to be gentle enough for use around the eye area (avoiding potential irritants like strong fragrance or menthol). The user can tap a small amount around the orbital bone to moisturize that delicate area. The cream's rich hydration and inclusion of barrier lipids (like ceramides) can help prevent fine lines (crow's feet) and dryness around the eyes, much as a dedicated eye cream would. If a specialized active like a peptide for eyes (e.g., a peptide that targets dark circles or puffiness) is desired, it can be included so that the cream doubles as an eye treatment. By covering this need, the system further negates the necessity of a separate eye cream step at night.

### Example Formulation—Step 3 Barrier Repair Cream

An exemplary formulation might be as follows, although higher or lower amounts may also be used depending on formulation needs:

| Category | Illustrative % Range |
|---|---|
| Vehicle/solvent | 45-60% |
| Humectants | 10-15% |
| Emollients/oils/esters (non-occlusive) | 10-15% |
| Occlusives (butters) | 5-12% |
| Barrier-support lipids/lamellar helpers | 1-3% |
| Soothing/probiotic & post-biotic ferments | 1-3% |
| Functional actives | 2-4% |
| Emulsifiers & texture/rheology modifiers | 3-6% |
| Preservative system & chelator | ≤1% |
| Botanical antioxidant/omega source | 0.5-1% |

This formulation yields a rich cream that, when applied, forms a semi-occlusive layer. To use, the individual takes a nickel-sized dollop and gently massages or pats it over the face (and neck if desired) as the final step at night. The skin may have a slight sheen initially (from the occlusives like shea butter and botanical oils (such as squalane or sunflower seed oils), which is indicative of the protective layer-akin to the effect of an overnight mask. By morning, the skin typically feels noticeably softer, deeply hydrated, and calm, with no greasy residue as the actives have absorbed and the occlusives have done their job preventing dehydration overnight. Studies have shown that such occlusive night treatments can result in plumper, less wrinkled skin by morning due to the intensive hydration and reduced trans-epidermal water loss. The inclusion of ceramides and related lipids means that, over days and weeks, the skin's baseline barrier function improves—users often report less dryness or sensitivity after adopting such a barrier-focused cream nightly. By serving as a moisturizer+night mask+barrier repair treatment in one, this Step 3 cream obviates the need for separate night treatments. If used in the morning, a smaller amount can be applied to avoid excessive shine, and it should be followed by sunscreen (since sun protection is always recommended, though not provided by the three-step kit itself).

In summary, the Barrier Repair Cream completes the regimen by sealing in the benefits and ensuring sustained hydration, which is critical for achieving the lustrous, smooth skin associated with multi-step routines. It distinguishes from conventional lightweight lotions by its emphasis on barrier occlusion and repair, an approach backed by dermatological science (occlusives+barrier lipids=improved skin moisture and integrity). The result is that even with only three products, the user's skin receives round-the clock care: cleansing without stripping, potent hydration and treatment, and thorough overnight nourishment/protection.

### Mapping of 3—Step System to Traditional Multi-Step Routines

To clearly illustrate how the invented regimen in certain embodiments substitutes for an 8-10 step routine, the following mapping is provided. Each step of the three-step system according to certain embodiments is correlated to the traditional steps it replaces:

Step 1 Cleanser-Replaces the double cleansing steps (oil-based cleanser+water-based cleanser) of a typical 10-step routine. It also initiates gentle exfoliation (replacing a separate exfoliator step, if one was used daily or almost daily). In essence, Step 1 covers what would be Step 1, 2, and partly Step 3 in a K-beauty routine (first cleanse, second cleanse, and some exfoliation).

Step 2 Treatment Essence-Replaces multiple middle steps: toner, essence, and serums/ampoules. In a 10-step routine, after cleansing, one might apply a toner (step 4) to hydrate and prep, then an essence (step 5) for additional hydration/nutrients, then one or more serums or ampoules (step 6, and possibly 7 if including treatments) for targeted concerns. The treatment essence herein combines these into one fluid. It hydrates like a toner/essence and delivers actives like a treatment serum. Additionally, by providing deep hydration, it can stand in for a face mask (which is often step 7)—many face masks are essentially delivering an “essence” to skin; essence according to certain embodiments can do so without the sheet. Thus, Step 2 effectively compresses steps 4 through 7 of the conventional routine.

Step 3 Barrier Repair Cream-Replaces the final moisturizing and sealing steps: moisturizer (day or night cream), facial oil, and the overnight sleeping mask (which some routines count as a separate step especially in the nighttime regimen). In Korean routines, a moisturizer is typically step 9 and an occlusive sleeping pack might be step 10 at night. The barrier repair cream serves both roles in certain embodiments—it is a robust moisturizer and functions as the sleeping mask to “seal it all in” as the ultimate protector.

Furthermore, it can double as an eye cream, since it is formulated gently but effectively for the eye area (replacing step 8, eye cream, in many routines). The cream's occlusive, intensive nature ensures the benefits of all prior steps are locked in, analogous to finishing a long routine with a heavy cream or mask to wake up with glowing skin.

Sun Protection (External to Kit)—The three-step system itself does not include a sunscreen in certain embodiments (as it focuses on core skincare), but it positions the skin such that adding a sun protection factor (SPF) component in the daytime is seamless. In a daily regimen, after the barrier repair cream in the morning (applied sparingly), a user would apply a broad-spectrum sunscreen (SPF 30+). Traditional routines consider sunscreen an essential final step in the morning. The absence of an SPF in this kit is not because it's unimportant-rather, sunscreen can be any standalone product of choice. In fact, SPF added into skincare products is often not sufficient for complete sun protection, as people rarely apply enough to achieve full coverage. The core three steps ensure the skin is well-treated; sun protection can be layered on top. (In an alternative embodiment, an AM version of Step 3 could incorporate SPF, though for flexibility and global considerations, a separate sunscreen is often preferable.)

This mapping demonstrates that the 3-step system indeed covers the gamut of an 8-10 step routine's functions with only three products. Importantly, it does so by multi-tasking—each product is somewhat “hybrid” in its benefits. Unlike a travel kit that might just include a cleanser, a toner, and a cream (requiring the user to accept skipping other steps or add their own serums), the disclosed system's products are formulated to ensure nothing critical is lost in consolidation. For example, while a basic three-step routine (cleanse, moisturize, protect) covers cleansing and hydration, it might not provide exfoliation or specialized treatment. The three-step system according to certain embodiments, by contrast, intentionally incorporates mild exfoliants and actives into its three steps, thereby addressing even concerns like dullness, acne management, or anti-aging, which simpler routines might neglect. This means users do not face the drawbacks some minimalist routines have (like not adequately targeting specific issues). Here, the minimalist approach is “strategically maximal”-minimal steps, maximal combined benefits.

From a user's perspective, the routine is dramatically simplified: rather than juggling a dozen bottles, they have three. This fosters consistency (users are more likely to stick to it) and reduces error (less chance of layering something incorrectly or overusing actives). Time-wise, it only requires a few minutes in the morning and at night, versus potentially half an hour each time with a complex routine. Cost-wise, it is also economical: instead of buying 8-10 products, one buys three, possibly with premium ingredients concentrated in them. This aligns with one of the benefits of a minimalist approach noted in skincare communities: saving money while still focusing on quality outcomes.

### Advantages Over Conventional Kits or Bundles

It is important to distinguish the present three-step system from existing multi-product kits that might superficially appear similar (e.g., a kit with three items). Many brands sell “starter sets” or “travel kits” that contain a few miniaturized products (cleanser, toner, lotion, etc.). However, those are different from the disclosed invention in certain embodiments:

Integrated Multifunction vs. Assorted Single-function: In the three-step system according to certain embodiments, each product is integrated and multifunctional, whereas in most kits the products are simply a smaller collection of the brand's standard single-function offerings. For example, a travel kit might have a tiny cleanser, a tiny toner, a tiny serum, and a tiny cream, each doing its narrow job. The three-step system according to certain embodiments has each step doing the work of three steps. This means if a conventional kit has 5 items, our system can achieve more with 3, as described above. No known travel set or mini kit focuses on collapsing functions; they typically aim to provide the full sequence in portable form, not to reduce the sequence. One commercial example touts an “all-in-one skincare kit delivering a complete routine” and that it “eliminates the need for multiple products” for travel, but in reality it still contains five separate formulas for five steps (cleanser, cream, sunscreen, etc.)—it eliminates carrying full size bottles, yet the user still must apply multiple products. In contrast, our system truly eliminates extra products by consolidating roles. This is a non-obvious improvement in the context of skincare kit design.

Synergistic Formulation: The three products in this system are formulated to complement each other when used sequentially. For instance, the slightly acidic nature of the cleanser and essence is calibrated so they work optimally one after another (no pH shock to skin, no residue from cleanser that could interfere with essence absorption). The barrier cream is designed to go on top of the essence without pilling (it has enough humectant compatibility and not too much silicone to cause rolling). In random kits, since products are often just a bunch of existing items thrown together, they may not layer well or could even have redundant ingredients without synergy. The three-step system according to certain embodiments from the ground up was developed as a unified regimen, which is a key distinction.

Educational Component: The kit is provided typically with instructions or a guide emphasizing the three-step usage and how it replaces other routines. This clarity helps users transition from complicated routines to this simplified one. Conventional kits sometimes lack clear guidance beyond “use these minis in your routine,” which can be confusing. By contrast, the inventive kit, in certain embodiments, teaches the user that these three are all they need. It promotes a mindset shift consistent with skinimalism—focusing on essentials of skin health (cleanse, hydrate/treat, protect) rather than chasing numerous steps.

Outcomes and “Glass Skin” Simplification: A major advantage is that the user can achieve trendy skincare results (like glass skin) more easily. Normally, attaining such highly hydrated, smooth skin requires dedication to layering multiple hydrating and exfoliating steps. Here, because each formula is power-packed (e.g., the essence deeply hydrates and exfoliates gently, the cream super hydrates and plumps overnight), the cumulative effect is akin to an 8-step moisture sandwich, but delivered in 3 layers. This means even a busy individual can realistically follow the routine and see results, whereas many people abandon 10-step routines due to time constraints or overwhelm. The invention, in certain embodiments, thus democratizes the results-making high-level skincare outcomes attainable without an elaborate process.

### Formulation Considerations for Sensitive and Acne-Prone Skin

One of the objectives of the invention, in certain embodiments, is to be inclusive of various skin types, including sensitive and acne-prone skin, while maintaining core functionality. Key formulation considerations and optional embodiments in this regard include:

Sensitive Skin: All three steps are formulated to minimize irritation. This means:

Using gentle surfactants (as mentioned, no sulfates; also avoiding known irritants like high concentrations of essential oils, menthol, denatured alcohol, etc.).

Keeping the formulations free of synthetic fragrances or with very low, allergen-screened fragrance. Fragrance is a common irritant for sensitive individuals, so a truly sensitive-skin version of the system would be fragrance-free. Any necessary scent can be from natural extracts with skin benefits (like a touch of chamomile for calming scent and effect). In certain embodiments, the formulations are fragrance-free, with no added synthetic or natural fragrances. Any subtle scent may come from functional natural extracts already included for skin benefits, such as chamomile or green tea.

Incorporating anti-inflammatory ingredients across the board: e.g., the cleanser with panthenol and allantoin, the essence with Centella and green tea, the cream with bisabolol and oat extract. In certain embodiments, the essence may include Centella, green tea, and Angelica root extract for soothing and antioxidant support, and the cream may include madecassoside, panthenol, and probiotic ferments to calm, repair, and strengthen sensitive skin. These help quickly soothe any potential irritation from cleansing or actives.

Patch-test friendly formulation: The products can be tested on extremely sensitive or eczema-prone skin in trials to ensure they do not cause redness or stinging. The absence of harsh exfoliants or at least the availability of an acid-free essence option is important: for very reactive skin, one embodiment of the essence contains no AHA/BHA, focusing purely on hydration and niacinamide (which is typically well-tolerated up to moderate percentages).

pH balancing: The cleanser's low pH itself helps sensitive skin by not disrupting the acid mantle; the essence around pH 5.0 is within skin's physiological range, which is ideal for barrier function.

Acne-Prone Skin: A concern with combining multiple functional steps into fewer formulations is whether the system might overload acneic or oily skin. In certain embodiments, the invention mitigates this by tailoring the compositions toward non-comedogenic, lightweight, and barrier-supportive ingredient sets.

Ensuring non-comedogenic ingredients: e.g., using light, non-pore-clogging oils (such as sunflower seed oil or squalane, which mimic natural sebum without congestion). Suitable occlusives include dimethicone and related silicones (e.g., cyclopentasiloxane), hydrogenated polyisobutene, caprylic/capric triglyceride-ingredients that are generally non-comedogenic when used at balanced levels. Still, the concentration of such occlusives in a day-use scenario can be moderated to avoid a heavy feeling.

Including anti-acne actives in a gentle way: In certain embodiments, the Step 1 cleanser may incorporate gentle AHA (Alpha Hydroxy Acid) together with betaine salicylate, a milder beta-hydroxy acid commonly used in K-beauty systems, to promote daily pore clarity without excessive irritation. The Step 2 treatment essence may provide niacinamide at about 4%, effective to reduce sebum production and soothe inflammation, although higher or lower amounts may also be used depending on formulation needs. Where further support is desired, the essence or cream may optionally include Centella asiatica-derived actives such as madecassoside, a gentle azelaic acid derivative, tea tree extract, or zinc PCA. In some embodiments, retinoids such as retinal or retinol may be excluded from the baseline system to reduce risk of irritation, but users may still incorporate prescription retinoids or other acne medications externally. The system is configured to be synergistic with such external additions; for example, a topical acne cream may be applied after the essence and before the cream, with the barrier cream helping to offset acne and barrier disruption commonly caused by such treatments.

Matte finish option: Acne-prone and oily individuals often dislike a greasy finish. The Step 3 cream for them can be made with more of a gel-cream consistency (as noted) and maybe including porous silica powder or polymethyl silsesquioxane plus zinc PCA (~0.25%) for lightweight sebum control and antibacterial benefit.

Avoiding comedogenic thickeners: Oily-skin targeted barrier creams or gel-creams often minimize waxes or heavy fatty alcohols and instead rely on silicone-based or polymer-based thickeners to achieve a lightweight, non-comedogenic texture.

The end result is a set that even those with acne can use to improve their skin's hydration and barrier. Often acne treatments dry out the skin, and a compromised barrier can actually worsen acne. By focusing on barrier repair, the system can assist acne patients in tolerating their treatments and maintaining balanced skin—this is a unique angle, as many acne kits are harsh (benzoyl peroxide washes, etc.), whereas this one is gentle and healing, which can complement active therapies.

Combination/Normal Skin: For those in between, the default formulations given will likely suit them, perhaps using moderate amounts of each product. The system inherently balances oil and hydration—the essence provides water, the cream provides oil/lipid-which combination skin can adjust by applying a bit less cream on oily areas, more on dry patches. The versatility of the kit allows personalization without extra products: e.g., a user with combination skin could use the cream primarily at night, and in the morning use just a tiny dab or even skip it if they feel the essence was enough under sunscreen for daytime. The instructions can mention these tweaks.

Adjustable Strength of Actives: The system can come in variations. For example, a gentle variation may have no acids and lower actives (for extremely sensitive), while an active variation might have a higher percentage of certain actives (like 10% vitamin C, or include a mild retinoid in the cream for an aging-focused set). This modular approach allows addressing specific concerns (aging, hyperpigmentation, etc.) by swapping the content of Step 2 or Step 3 primarily, while keeping the 3-step structure constant. Importantly, even in an “active” version, the barrier focus remains (the cream still has occlusives and ceramides) to ensure tolerability.

By accounting for sensitive and acne-prone skin in the design, the invention, in certain embodiments, stands out as an inclusive minimal regimen. Many minimal routines fail certain groups (e.g., overly basic for acne skin, or still irritating for super sensitive skin because they consolidate by removing beneficial soothing steps). Here, we consolidate by integrating soothing and treatment into the steps, not by leaving them out. This means even those with previously high-maintenance skin conditions can attempt to downsize their routine to these three products and still see improvements, not flares. Dermatologists often emphasize gentle cleansing, basic moisturizing, and sunscreen as the essentials for problem skin; this system adheres to that but goes a step further to deliver targeted benefits, fulfilling both dermatological and beauty goals.

### Embodiments and Example Use Scenarios

To further illustrate aspects and embodiments of the invention, some exemplary scenarios are described:

Example 1 (General User, Normal Skin): A user with normal, mildly dehydrated skin adopts the three-step system according to certain embodiments. In the morning, she starts with the Step 1 cleanser to refresh her skin and remove any overnight sweat or oil. She pats on Step 2 essence, which absorbs quickly and leaves her skin dewy. She follows with a light layer of Step 3 cream and then her own sunscreen. Her makeup (if any) goes on smoothly over this. In the evening, she double cleanses using only the Step 1 cleanser, massaging it into dry skin to melt away sunscreen and makeup, then rinsing thoroughly. If she wore heavier makeup that day, she simply uses a bit more product and spends extra time massaging it in to ensure a complete cleanse. She applies the essence again (sometimes twice for extra hydration), then a thicker layer of the barrier cream as an overnight mask. After two weeks, she notices her skin looks as glowing and moist as it did when she tried a 8-step routine before, but now with far less hassle. Her fine dry lines are plumped, and she hasn't experienced the mild irritation she used to get when layering many acids and serums. She's pleased that a simpler routine gave equal or better results, confirming the system's efficacy.

Example 2 (Oily, Acne-Prone Skin): A male user with oily, acne-prone skin tries the system (oily-skin variant). The Step 1 cleanser keeps his pores clear and reduces oiliness without needing a separate medicated cleanser. The Step 2 essence provides hydration with niacinamide and green tea, which actually calms his breakouts and redness; it's light enough that he doesn't feel greasy. Initially, he is hesitant about the Step 3 cream (worried it might be too heavy), but the gel-cream version sinks in nicely. He notices that by repairing his skin barrier at night with the cream, the usual dryness and irritation from his prescription acne gel is reduced-his skin stays balanced (not too oily, not flaky). Over time, his breakouts diminish in severity, partly because his skin is not over-stripped by harsh products anymore and the consistent mild exfoliation prevents clogged pores. This demonstrates how even acne-prone individuals can benefit from a barrier centric approach.

Example 3 (Busy Mom vs. At-Home): A frequent traveler and busy mom carries the three-step kit according to certain embodiments instead of a bag full of products. The compact three bottles save space, and because each performs multiple tasks, she doesn't feel she's compromising her routine on the road. She notes that while previously travel or daily chaos would wreak havoc on her skin (due to either skipping steps or dry airplane environments), now the barrier cream keeps her skin hydrated on flights (she even applies a bit mid-flight as a mask), and the simplified routine is easy to do even when exhausted or on-the-go. This scenario highlights the practicality and commercial appeal of the system as a travel-friendly solution that is actually effective, not just convenient.

These scenarios underscore the present invention's ability, in certain embodiments, to simplify without sacrifice. Traditional wisdom might suggest you need many products for such results, but aspects of the present invention refutes that, showing synergy and smart formulating can achieve the same.

### Illustrative Ingredient Variations by Skin Type

In certain embodiments, the compositions disclosed herein are adaptable across skin types through strategic ingredient variations in each of the three product formulations. While the structural and functional roles of Step 1 (multi-purpose cleanser), Step 2 (treatment essence), and Step 3 (barrier repair cream) remain constant across all versions, the choice and concentration of specific ingredients may be tailored to address the differing needs of sensitive, oily/acne-prone, dry/barrier-damaged, aging, uneven skin toned/hyperpigmentation, and combination/normal skin types.

For sensitive skin, the Step 1 multi-purpose cleanser is formulated with or without exfoliants or strong surfactants and instead includes ingredients such as chamomile extract, panthenol (pro-vitamin B5), and allantoin to soothe and minimize irritation. The Step 2 treatment essence avoids direct acids (e.g., AHA, BHA) and may include calming ingredients such as niacinamide (at ≤3%), Centella asiatica, oat extract, and beta-glucan, all known for their barrier-supportive and anti-inflammatory properties. Fragrance is excluded in sensitive-skin embodiments to minimize allergic or irritant reactions. The Step 3 cream for sensitive users contains ceramides and cholesterol for lipid replenishment, panthenol for repair, and oat-derived polysaccharides for anti-redness support, again avoiding fragrances and potentially irritating actives like menthol or retinol derivatives.

For oily or acne-prone skin, the Step 1 cleanser may include oil-control and pore-refining actives such as 0.5% salicylic acid, green tea extract, and optionally tea tree oil. The base is a non-comedogenic gel or gel-cream to ensure effective cleansing without clogging pores. The Step 2 essence may include 4-5% niacinamide for sebum regulation, zinc PCA for antimicrobial and oil-balancing effects, and green tea extract for inflammation control. A gentle exfoliant such as mandelic or lactic acid may be present at low concentration to promote cell turnover without over-stripping. The Step 3 barrier cream for oily or acne-prone skin is typically a lightweight gel-cream formulation with a lower total oil phase. Emollients may include dimethicone or other silicones for a breathable, matte finish, and the formula may include silica or zinc PCA to reduce surface oil and provide antibacterial benefits.

For dry or barrier-damaged skin, the Step 1 cleanser may be optimized for hydration and comfort. It may include increased levels of glycerin and panthenol and avoids acids or alcohol. The Step 2 treatment essence may include a blend of high- and low-molecular-weight hyaluronic acid for deep and surface hydration, urea (3-5%) for both humectant and mild keratolytic activity, and ceramide NP to reinforce the lipid matrix. Mild peptides and saccharide isomerates may also be included to increase moisture retention. The Step 3 barrier repair cream in this embodiment may include 5-15% shea butter or cocoa butter, 5-10% squalane or triglyceride-rich oils, 2% or more of a ceramide complex, and optionally 5-10% petrolatum or lanolin to create a lasting occlusive seal. This version is designed to rebuild compromised barriers and prevent trans-epidermal water loss overnight.

For combination or normal skin, a balanced formulation may be used. The Step 1 cleanser may employ a blend of gentle surfactants with optional inclusion of an enzyme-based exfoliant (such as papain or bromelain) for light daily resurfacing. The Step 2 treatment essence includes a core set of humectants (glycerin, butylene glycol), 2-4% niacinamide, and a small amount of a mild acid (e.g., 1-2% lactic acid) for smoothing and brightness. The Step 3 cream balances occlusives and emollients for a mid-weight feel-enough to seal in moisture but light enough for daily wear across all zones of the face. A core lipid blend of ceramides, cholesterol, and fatty acids supports the barrier while avoiding overly rich or overly matte finishes. This version is versatile, and in many cases serves as the foundation for the other skin-type variants by adjusting the proportions of key ingredients.

In certain embodiments, the formulations are preferably pH-balanced, non-comedogenic, and free of known irritants, with optional fragrance or essential oils included only at low levels and with labeling variants indicating fragrance-free versions. Each formulation retains the multifunctional character of its respective step, ensuring that even with ingredient customization, the overall three-step routine delivers cleansing, hydration, treatment, and occlusive protection comparable to a full-length multi-step regimen.

While the above description contains many specifics and certain exemplary embodiments have been described, it is to be understood that such embodiments are merely illustrative of, and not restrictive on, the broad invention, and that this invention is not to be limited to the specific constructions and arrangements shown and described, since various other modifications may occur to those ordinarily skilled in the art, as mentioned above. The invention includes any combination or sub-combination of the elements from the different species and/or embodiments disclosed herein.

What is claimed is:

1. A skincare composition for cleansing, comprising:
one or more gentle surfactants in an amount effective to remove oil-based and water-based impurities from the skin;
one or more emulsifiers to emulsify makeup, sebum, and sunscreen on the skin upon contact with water;
one or more humectants at approximately 5% to 20% by weight;
one or more exfoliating agents selected from beta hydroxy acids, alpha hydroxy acids, polyhydroxy acids, enzymes, or gentle scrubbing particles in an amount sufficient to provide mild exfoliation without irritation;
one or more skin-soothing agents; and
one or more barrier-supporting agents selected from ceramides, ceramide derivatives, phospholipids, probiotic ferment filtrates, or film-forming polymers;
wherein the composition is sulfate-free and PH balanced and, upon application, removes both oil-soluble and water-soluble debris in a single step.

2. The composition of claim 1, wherein the cleanser transforms from a balm, gel, oil, cream, powder, or milk into a foam or milky emulsion upon contact with water.

3. The composition of claim 1, wherein the exfoliating agent comprises encapsulated scrubbing particles that rupture upon hydration or mechanical action.

4. The composition of claim 1, wherein the composition has a pH of 4.5 to 6.0.

5. A multifunctional treatment essence composition, comprising:
an aqueous carrier with one or more humectants present at 5% by weight or greater;
one or more active treatment ingredients selected from niacinamide, vitamin C, vitamin E, peptides, alpha hydroxy acids, beta hydroxy acids, polyhydroxy acids, or botanical extracts;
one or more barrier-supporting agents selected from ceramides, ceramide derivatives, phospholipids, probiotic ferment filtrates, or film-forming polymers; and
one or more soothing agents;
wherein the composition is a lightweight liquid or gel that is rapidly absorbable and is configured to provide the combined functional benefits of a toner, an essence, and a serum.

6. The composition of claim 5, wherein the active comprises niacinamide at approximately 2% to 5% by weight.

7. The composition of claim 5, wherein the active treatment ingredient comprises a polyhydroxy acid selected from gluconolactone or lactobionic acid at approximately 0.5% to 3% by weight.

8. The composition of claim 5, wherein the composition substitutes for toner, serum, and a sheet mask by hydrating, delivering actives, and providing barrier repair in a single product.

9. A barrier repair cream composition, comprising:
an emulsion of water and lipids, the lipid phase including one or more occlusive or emollient agents selected from petrolatum, dimethicone, hydrogenated polyisobutene, lanolin, natural butters, or plant-derived oils in an amount of 5% to 40% by weight sufficient to reduce trans-epidermal water loss;
a barrier-supporting system comprising one or more components selected from ceramides, ceramide derivatives, phospholipids, lamellar emulsifiers, cholesterol, or fatty acids;
one or more humectants at approximately 2% to 20% by weight; and
one or more additional actives selected from peptides, vitamins, antioxidants, probiotic ferment filtrates, or soothing botanical extracts;
wherein the composition, when applied as a final skincare step, forms a protective moisturizing film that remains effective on the skin for at least 6 hours, functioning as both a moisturizer and an overnight mask.

10. The composition of claim 9, wherein the lipid phase comprises rice bran oil at approximately 0.5% to 5% by weight or squalane at approximately 1% to 8% by weight.

11. The composition of claim 9, wherein the barrier-supporting system comprises ceramide NP, ceramide AP, or ceramide EOP in combination with stearic acid and cholesterol in a weight ratio of approximately 2:1:1 to 1:1:1.

12. The composition of claim 9, wherein application reduces trans-epidermal water loss by at least 20% after 8 hours.

13. The composition of claim 9, wherein the additional active comprises niacinamide at approximately 2% to 5% by weight.

14. The composition of claim 9, wherein the additional active comprises a peptide selected from palmitoyl tripeptide-5, acetyl hexapeptide-8, or copper peptide.

15. The composition of claim 9, wherein the composition has a pH of 4.5 to 6.0.

16. A skincare kit comprising:
a first container containing the cleanser composition of claim 1;
a second container containing the essence composition of claim 5;
a third container containing the barrier repair cream composition of claim 9; and
instructional material identifying the products as Step 1, Step 2, and Step 3 in a sequential regimen;
wherein the cleanser prepares the skin for the essence, the essence primes the skin for the barrier cream, and the barrier cream seals in the benefits of the prior steps to provide cumulative effects comparable to a multi-product skincare routine with fewer products.

17. The kit of claim 16, wherein the cleanser is the composition of claim 2.

18. The kit of claim 16, wherein the cleanser is the composition of claim 3.

19. The kit of claim 16, wherein the cleanser is the composition of claim 4.

20. The kit of claim 16, wherein the essence is the composition of claim 6.

21. The kit of claim 16, wherein the essence is the composition of claim 7.
